(19) United States
(12) Patent Application Publication
(10) Pub. No.: US 2026/0355223 A1
(43) Pub. Date: Nov. 12, 2026

(54) METHODS FOR TREATMENT OF COCAINE USE DISORDER

(71) Applicant: THE UAB RESEARCH FOUNDATION, Birmingham, AL (US)

(72) Inventor: Peter S. Hendricks, Birmingham, AL (US)

(21) Appl. No.: 19/674,573

(22) Filed: May 12, 2026

Related U.S. Application Data
(60) Provisional application No. 63/804,162, filed on May 12, 2025.

(51) Int. Cl. A61K 31/675; A61P 25/30

(52) U.S. Cl. CPC A61K 31/675; A61P 25/30

(57) ABSTRACT

In one aspect, the disclosure relates to a method for the treatment of cocaine use disorder in a mammal comprising at least the step of administering to the mammal a therapeutically effective amount of psilocybin or a pharmaceutical composition thereof. Also disclosed herein are kits associated with the treatment of cocaine use disorder. This abstract is intended as a scanning tool for purposes of searching in the particular art and is not intended to be limiting of the present disclosure.

### CROSS-REFERENCE TO RELATED APPLICATIONS

This application claims the benefit of and priority to U.S. Provisional Application No. 63/804,162, filed on May 12, 2025 which is incorporated herein by reference in its entirety.

### BACKGROUND

Global cocaine trafficking and use is at an all-time high and on the rise. Cocaine use is more likely to elicit dependence than other substances such as alcohol and cannabis. Whereas pharmacotherapies have been developed for several substance use disorders, medications for stimulant use disorders remain elusive. Therefore, there is a need for effective treatments for cocaine use disorder.

### SUMMARY

In accordance with the purpose(s) of the disclosure, as embodied and broadly described herein, the disclosure, in one aspect, relates to methods of treating cocaine use disorder.

Disclosed are methods for the treatment of cocaine use disorder in a mammal comprising the step of administering to the mammal a therapeutically effective amount of psilocybin or a pharmaceutical composition thereof. Also disclosed herein are methods for the treatment of cocaine use disorder in a mammal comprising: a) administering to the mammal a therapeutically effective amount of psilocybin or a pharmaceutical composition thereof; and b) providing the mammal with psychological support.

Also disclosed are uses of a disclosed compound, or a pharmaceutically acceptable salt, a hydrate, a solvate, or a polymorph thereof, in the manufacture of a medicament for the treatment of cocaine use disorder in a mammal.

Also disclosed is a kit comprising a therapeutically effective amount of psilocybin or a pharmaceutical composition thereof; and one or more of: a) at least one agent known to treat a cocaine use disorder; or b) instructions for treating a cocaine use disorder, comprising instructions for providing psychological support.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described aspects are usable in all aspects of the disclosure taught herein. Furthermore, the individual features of the dependent claims, as well as all optional and preferred features and modifications of the described aspects are combinable and interchangeable with one another.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1D show a Trial Profile, also known as a CONSORT Flow Chart, demonstrating participant flow throughout a representative trial.

FIG. 2A shows a mean percentage of cocaine abstinent days at various time periods for participants randomized to psilocybin and placebo for a representative trial across the entire study period by treatment condition. Error bars indicate ±1 standard error of the mean. Note that assessment windows differ for each time period, with approximately 30 days for prescreening and preparation, approximately 40 days (placebo) and 65 days (psilocybin) for integration, and 90 days for baseline, day 90, and day 180.

FIG. 2B shows Kaplan-Meier curves for participants randomized to psilocybin and placebo for a representative trial. A reduced risk of cocaine lapse in participants who receive psilocybin, as compared with the placebo, is shown. Shaded areas around the lines indicate 95% confidence intervals.

FIG. 3 shows effects of psilocybin treatment on meaning in life for participants with cocaine use disorder relative to a placebo in a representative trial.

FIG. 4 shows effects of psilocybin treatment on Machiavellianism for participants with cocaine use disorder relative to a placebo in a representative trial.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual aspects described and illustrated herein has discrete components and features which may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

Reference to “a” chemical compound refers to one or more molecules of the chemical compound rather than being limited to a single molecule of the chemical compound. Furthermore, the one or more molecules may or may not be identical, so long as they fall under the category of the chemical compound. Thus, for example, “a” chemical compound is interpreted to include one or more molecules of the chemical, where the molecules may or may not be identical (e.g., different isotopic ratios, enantiomers, and the like).

As used in the specification and the appended claims, the singular forms “a,” “an” and “the” include plural referents unless the context clearly dictates otherwise. Thus, for example, reference to “an excipient,” “a carrier,” or “a session” includes, but is not limited to, two or more such excipients, carriers, or sessions.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds, compositions, and the like for use in all of the major therapeutic areas including, but not limited to, adjuvants; anti-infectives such as antibiotics and antiviral agents; analgesics and analgesic combinations; anorexics; anti-inflammatories; anti-epileptics; local and general anesthetics; hypnotics; sedatives; antipsychotics; neuroleptics; antidepressants; anxiolytics; antagonists; neuron blocking; anticholinergics and cholinomimetics; antimuscarinics and muscarinics; antiadrenergics; antiarrhythmics; antihypertensives; hormones; nutrients; antiarthritics; antiasthmatics; anticonvulsants; antihistamines; antinauseants; antineoplastics; antipruritics; antipyretics; antispasmodics; antimicrobials; cardiovascular preparations (including calcium channel blockers, beta-blockers, beta-agonists and antiarrythmics); antihypertensives; diuretics; vasodilators; central nervous system stimulants; cough and cold preparations; decongestants; diagnostics; bone growth stimulants and bone resorption inhibitors; immunosuppressives; living therapeutics (e.g., altered or genetically engineered bacteria); muscle relaxants; psychostimulants; sedatives; tranquilizers; proteins, peptides, and fragments thereof (whether naturally occurring, chemically synthesized or recombinantly produced); nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like); probiotics such as bacteria and yeast; small molecules (e.g., diclofenac) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, troubleshooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and juvenile subjects, whether male or female, are intended to be covered. In one aspect, the subject is a mammal. A patient refers to a subject afflicted with a disease or disorder. The term “patient” includes human and veterinary subjects.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, “complete abstinence” in relation to a substance refers to a total absence of said substance use by a subject throughout the entire duration of a study or treatment period. For example, in a 12-week clinical trial for CUD, complete abstinence would mean a subject participating in the trial has no instances of cocaine use—confirmed, for example, by negative urine drug screens or self-reports—for the full 12 weeks.

As used herein, the term “cocaine use disorder” (abbreviated as CUD) refers to a pattern of cocaine use resulting in clinically significant impairment or distress. CUD can be defined as outlined in the Diagnostic and Statistical Manual of Mental Disorders (DSM), 5th edition, text revision (DSM-5-TR) or the DSM-IV (where it is labeled “cocaine dependence”). For example, a subject can be diagnosed with a CUD under the criteria outlined in the DSM-IV (labeled as cocaine dependence), under the criteria outlined in the DSM-5-TR (labeled as cocaine use disorder), or under the criteria of both.

The DSM-5-TR defines CUD as clinically significant impairment or distress that is caused by at least 2 of 11 identified criteria in the prior 12 months. Briefly, these 11 criteria can be organized into the following 4 groups: (1) physiologic (e.g., craving, tolerance, and withdrawal); (2) loss of control of cocaine use; (3) cocaine use taking precedence over other activities (e.g., personal responsibilities, such as at home, or professional responsibilities, such as at school or work); and (4) other negative consequences from cocaine use. The DSM further characterizes mild, moderate, and severe CUD.

DSM-IV defines cocaine dependence as a maladaptive pattern of cocaine use leading to clinically significant impairment or distress, manifested by three or more of seven specific criteria occurring within the same 12-month period. These criteria include tolerance, withdrawal, using larger amounts or over longer periods than intended, unsuccessful efforts to cut down, excessive time spent obtaining/using/recovering, giving up important activities, and continued use despite known physical or psychological problems caused by cocaine. This diagnosis is separate from (and more severe than) cocaine abuse in DSM-IV; the framework has been replaced in DSM-5 by a single “Cocaine Use Disorder” diagnosis.

Psychological support refers to the application of psychological methods directed towards a subject, where psychological methods are interventions that are delivered via various forms of communication and/or interaction (e.g., verbal, nonverbal, visual, physical). The application can be performed by a licensed mental health professional or by a non-licensed facilitator. Psychological support can be delivered with the intention to treat a psychological condition or can be delivered merely with the intention to maintain a subject's safety and/or to adhere to ethical standards in the context of psychedelic drug administration. Psychotherapy can be considered a type of psychological support.

As used herein, psychotherapy can refer to the application of psychological methods directed towards a subject and performed by a licensed mental health professional with the intention of assessing, diagnosing, and/or treating a psychological condition.

Unless otherwise specified, temperatures referred to herein are based on atmospheric pressure (i.e. one atmosphere).

### B. INTRODUCTION

Pharmacotherapies have been developed for the treatment of disorders such as opioid use disorder, alcohol use disorder, and tobacco use disorder. In comparison, medications for the treatment of stimulant use disorders, including CUD, remain elusive. Many different pharmacotherapy candidates have been targeted for the treatment of stimulant use disorders including bupropion, naltrexone, topiramate, antipsychotics, antidepressants, psychostimulants, as well as combinations of these medications. However, a recent meta-analysis of 157 studies examining 11 different treatment categories concluded that only contingency management, a behavioral intervention that faces notable barriers to implementation, is associated with increased abstinence from cocaine.

The success of a particular treatment for stimulant use disorders can be measured by several metrics, including outcomes such as complete abstinence following treatment. Complete abstinence is a stringent measure, often seen as the gold standard in addiction research because it indicates a total cessation of use. However, it's also a high bar that many participants don't meet, especially in the context of stimulant use disorders where relapse rates are high. Complete abstinence doesn't allow for any lapses; a single positive drug test or reported use during the trial period would disqualify a participating subject from being classified as completely abstinent. Another metric of treatment success is treatment retention by a subject participating in treatment. Treatment retention is an important outcome, as withdrawing from treatment can be associated with a relapse to stimulant use.

Complete abstinence in particular has largely been abandoned as an outcome in CUD trials because so few people accomplish this outcome. Metrics such as intermittent abstinence, sustained abstinence, or reduction in use have been recommended as alternative indicators of treatment success. These measures can be useful means for evaluating treatment success, particularly in stimulant use cases where the stimulant poses the potential for serious harm to a user's health. However, complete abstinence remains one of the most desired outcomes for treating stimulant use disorders.

Psychedelics such as lysergic acid diethylamide (LSD) and psilocybin have demonstrated signals of transdiagnostic efficacy in the treatment of mental health conditions including depressive disorders and demoralization. In the 1960s LSD showed promise in the treatment of alcohol use disorder across several different clinical trials, and one study published in 1973 suggested LSD may be effective in treatment of opioid use disorder. No prior trials have evaluated psychedelics in the treatment of CUD.

In one aspect, disclosed herein are methods for the treatment of CUD comprising the administration of a therapeutically effective amount of psilocybin or a pharmaceutical composition thereof and kits comprising a therapeutically effective amount of psilocybin or a pharmaceutical composition thereof. The methods can further include providing psychological support. The disclosed methods and/or kits can be used in the process of treating CUD. The disclosed methods of treatment can result in a subject (that has been diagnosed with a need for treatment of CUD) undergoing the treatment to achieve more desirable outcomes compared to treatment via other existing methods of treating CUD. For example, subject(s) can achieve an outcome of complete abstinence via the methods disclosed herein. Additionally, subjects can achieve higher rates or likelihood of treatment retention, longer periods of sustained abstinence, and more frequent periods of abstinence (e.g., more days abstinent) compared to existing methods of treating CUD. Additionally, the disclosed methods for the treatment of CUD can prove effective in subjects from underrepresented and/or socioeconomically disadvantaged populations.

### C. PSILOCYBIN COMPOUNDS

In one aspect, the disclosure relates to psilocybin compounds and salts, hydrates, solvates, or polymorphs thereof. Psilocybin, also known as 4-phosphoryloxy-N,N-dimethyltryptamine can be found in and isolated from a variety of species of mushrooms, such as members of genera Psilocybe, Panaeolus, Inocybe, Pluteus, Gymnopilus, Galerina, and Pholiotina. Additionally, psilocybin can be synthetically produced via chemical synthesis or biosynthesis.

In various aspects, it is contemplated herein that the disclosed compounds further comprise their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, Sep. 10, 2011, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, it is contemplated herein that the disclosed compounds further comprise their isotopically-labelled or isotopically-substituted variants, i.e., compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### D. PHARMACEUTICAL COMPOSITIONS

In various aspects, the present disclosure relates to pharmaceutical compositions comprising a therapeutically effective amount of at least one disclosed compound (e.g., psilocybin) or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound or a pharmaceutically acceptable salt thereof as an active ingredient, a pharmaceutically acceptable carrier, a pharmaceutically acceptable excipient, optionally one or more other therapeutic agents, optionally one or more adjuvants, or a combination thereof. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a pharmaceutically acceptable salt, a hydrate thereof, a solvate thereof, a polymorph thereof, or a stereochemically isomeric form thereof. In a further aspect, a disclosed compound, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutically acceptable excipients and/or additives. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N, N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl) imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure can be in a form suitable for injection, such as parenteral administration (e.g., intravenous, intramuscular, or subcutaneous administration). Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long-acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

An appropriate dosage level will generally be about 0.01 to about 1.5 mg per kg patient body weight per administration and can be administered in single or multiple doses. In various aspects, the dosage level can be about 0.1 to about 1.5 mg/kg per administration, about 0.1 to about 1.4 mg/kg per administration, about 0.1 to about 1.3 mg/kg per administration, about 0.1 to about 1.2 mg/kg per administration, about 0.1 to about 1.1 mg/kg per administration, about 0.1 to about 1 mg/kg per administration, about 0.1 to about 0.9 mg/kg per administration, about 0.1 to about 0.8 mg/kg per administration, about 0.1 to about 0.7 mg/kg per administration, about 0.1 to about 0.6 mg/kg per administration, about 0.1 to about 0.5 mg/kg per administration, about 0.1 to about 0.4 mg/kg per administration, about 0.1 to about 0.3 mg/kg per administration, about 0.1 to about 0.2 mg/kg per administration, about 0.01 to about 1.5 mg/kg per administration, about 0.01 to about 1.4 mg/kg per administration, about 0.01 to about 1.3 mg/kg per administration, about 0.01 to about 1.2 mg/kg per administration, about 0.01 to about 1.1 mg/kg per administration, about 0.01 to about 1 mg/kg per administration, about 0.01 to about 0.9 mg/kg per administration, about 0.01 to about 0.8 mg/kg per administration, about 0.01 to about 0.7 mg/kg per administration, about 0.01 to about 0.6 mg/kg per administration, about 0.01 to about 0.5 mg/kg per administration, about 0.01 to about 0.4 mg/kg per administration, about 0.01 to about 0.3 mg/kg per administration, about 0.01 to about 0.2 mg/kg per administration, or about 0.01 to about 0.1 mg/kg per administration. In one aspect, for oral administration, the dosage range can be about 0.01 to about 1.5 mg/kg per administration, about 0.01 to about 1 mg/kg per administration, about 0.01 to about 0.5 mg/kg per administration, about 0.01 to about 0.1 mg/kg per administration, about 0.1 to about 1.5 mg/kg per administration, about 0.1 mg/kg to about 1 mg/kg per administration, or about 0.1 mg/kg to about 0.5 mg/kg per administration. For oral administration, the compositions are preferably provided in the form of tablets containing 1.0 mg to 100 mg of the active ingredient, particularly 0.5, 1.0, 2.0, 4.0, 6.0, 8.0, 10, 15, 20, 25, 50, 75, and 100 mg of the active ingredient for the symptomatic adjustment of the dosage of the patient to be treated.

In another aspect, an appropriate dosage level can be a flat dosage level, generally 5 to about 150 mg per administration and can be administered in single or multiple doses. In various aspects, the dosage level can be 5 mg to about 150 mg per administration, 5 mg to about 125 mg per administration, 5 mg to about 100 mg per administration, 5 mg to about 90 mg per administration, 5 mg to about 80 mg per administration, 5 mg to about 70 mg per administration, 5 mg to about 60 mg per administration, 5 mg to about 50 mg per administration, 5 mg to about 40 mg per administration, about 10 mg to about 150 mg per administration, about 10 mg to about 125 mg per administration, about 10 mg to about 100 mg per administration, about 10 mg to about 90 mg per administration, about 10 mg to about 80 mg per administration, about 10 mg to about 70 mg per administration, about 10 mg to about 60 mg per administration, about 10 mg to about 50 mg per administration, about 10 mg to about 40 mg per administration, about 15 mg to about 100 mg per administration, about 15 mg to about 80 mg per administration, about 15 mg to about 60 mg per administration, about 15 mg to about 50 mg per administration, about 15 mg to about 40 mg per administration, about 20 mg to about 100 mg per administration, about 20 mg to about 80 mg per administration, about 20 mg to about 60 mg per administration, about 20 mg to about 50 mg per administration, or about 20 mg to about 40 mg per administration. Within this range the dosage can be, for example, 5 mg to 25 mg, 10 mg to 30 mg, 15 mg to 35 mg, 20 mg to 40 mg, or 25 mg to 40 mg per administration. In one aspect, for oral administration, the dosage range can be 5 mg to about 100 mg per administration, 5 mg to about 100 mg per administration, 5 mg to about 90 mg per administration, 5 mg to about 80 mg per administration, 5 mg to about 70 mg per administration, 5 mg to about 60 mg per administration, 5 mg to about 50 mg per administration, 5 mg to about 40 mg per administration, about 10 mg to about 100 mg per administration, about 10 mg to about 90 mg per administration, about 10 mg to about 80 mg per administration, about 10 mg to about 70 mg per administration, about 10 mg to about 60 mg per administration, about 10 mg to about 50 mg per administration, or about 10 mg to about 40 mg per administration.

In another aspect, an appropriate dosage level can be smaller and include sub-sensorium (e.g., little to no experience of an altered state of consciousness by a subject) levels of administration (e.g., microdosing). In various aspects, the dosage level can be a flat dosage level, generally about 10 μg to less than 5 mg per administration. In various aspects, the dosage level can be about 10 μg to less than 5 mg per administration, about 10 μg to about 1 mg per administration, about 10 μg to about 750 μg per administration, about 10 μg to about 500 μg per administration, about 10 μg to about 250 μg per administration, or about 10 μg to about 100 μg per administration. In one aspect, the number of administrations can be more for lower dosage levels (e.g., microdoses) of the compound.

In one aspect, the compound can be administered a single time over the course of treatment. In another aspect, the compound can be administered multiple times over the course of treatment (e.g., on a regimen of 1 to 4 times (1 to 4 administrations) per month. This dosing regimen can be adjusted to provide the optimal therapeutic response. As one example, a typical dosage can be one about 1 mg to about 100 mg tablet/capsule, one about 1 mg to about 75 mg tablet/capsule, one about 1 mg to about 50 mg tablet/capsule, one about 1 mg to about 25 mg tablet/capsule, one about 1 mg to about 10 mg tablet/capsule, or one about 1 mg to about 5 mg tablet/capsule taken a single time, once a day, once a week, once a month, or multiple times per month, or one time-release capsule or tablet taken a single time, once a day, once a week, or once a month and containing a proportionally higher content of active ingredient. The time-release effect can be obtained by capsule materials that dissolve at different pH values, by capsules that release slowly by osmotic pressure, or by any other known means of controlled release.

Such unit doses as described hereinabove and hereinafter can be administered a single time over the course of treatment or multiple times in a month, for example, 2, 3, 4, 5, or 6 times a month. In various aspects, such unit doses can be administered 1 or 2 times per day, so that the total dosage for a 70 kg adult is, for example, in the range of 0.005 to about 15 mg per kg weight of subject per administration. In a further aspect, dosage is 0.01 to about 1.5 mg per kg weight of subject per administration, and such therapy can extend for a number of weeks or months, and in some cases, years. Smaller unit doses can be administered once a day or multiple times in a week, for example 2, 3, 4, 5, or 6 times a week, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

### E. METHODS OF USING THE COMPOUNDS

In one aspect, disclosed herein are uses of psilocybin or a pharmaceutically acceptable salt, a hydrate, a solvate, or a polymorph thereof in the manufacture of a medicament for the treatment of CUD in a mammal.

In another further aspect, the present disclosure provides methods of treatment of CUD comprising administration of a therapeutically effective amount of a disclosed compound (e.g., psilocybin) or pharmaceutical composition as disclosed herein above to a subject in need thereof. The methods can further comprise providing psychological support.

Administration of psilocybin can be marked by a subject experiencing an altered state of consciousness, which can be indicated by alterations in a subject's perceptions, mood, and/or thought and/or alterations in a subject's experience of their environment (including time and space). Examples of these effects can include physiologic effects (e.g., elevation in body temperature, yawning, feeling of weightlessness), visual effects (e.g., color saturation enhancement, perspective distortion), cognitive effects (e.g., increased empathy, ego loss), auditory effects (e.g., sound enhancement), multisensory effects (e.g., synesthesia), and transpersonal effects (e.g., sense of interconnection between humanity and a higher power). These effects can be referred to as the initial effects of psilocybin administration. These effects may not occur immediately after administration. Depending on the method of administration, a subject may not feel the initial effects of psilocybin until, for example, about 10 minutes to about 1 hour after administration. A subject can experience the initial effects of psilocybin for a period of, for example, about 1 hour to about 7 hours. This period can be broken up into an early stage of initial effects, where the effects begin and are increasing in intensity (lasting, for example, about 10 minutes to about 1 hour); a middle stage of initial effects, where the initial effects are at their strongest and maintain a relatively steady intensity (lasting, for example, about 1 hour to about 4 hours); and a late stage of initial effects, where initial effects are decreasing in intensity and end (lasting, for example, about 30 minutes to about 3 hours). Later effects of psilocybin administration can occur, for example, from about 1 hour to about 24 hours after administration or after the late stage of initial effects is over.

In one aspect, the method of treatment can further comprise psychological support (e.g., psychotherapy). This psychological support can be provided in a controlled environment. A subject can participate in a psychological support session prior to administration or post-administration of a therapeutically effective amount of a disclosed compound or pharmaceutical composition as disclosed herein. A subject can participate in one or more psychological support sessions prior to administration. A subject can participate in a psychological support session about 4 weeks, about 3 weeks, about 2 weeks, about 1 week, about 4 days, about 1 day, about 18 hours, about 12 hours, about 6 hours, about 4 hours, about 2 hours, about 1 hour, or about 30 minutes prior to administration of a therapeutically effective amount of a disclosed compound or pharmaceutical composition as disclosed herein.

A subject can participate in one or more psychological support sessions post-administration. The post-administration psychological support can include the subject participating in a psychological support session during any one of the stages of psilocybin effects (i.e., prior to the initial effects being experienced by the patient, during the early stage of initial effects, during the middle stage of the initial effects, during the late stage of initial effects, or after the end of the late stage of initial effects) and, optionally, extending into any subsequent stage (e.g., beginning a psychological support session during the middle stage of the initial effects and continuing into or ending the session in the late stage of initial effects). A post-administration support session occurring after the end of the late-stage initial effects can occur during the same day as administration, about 1 to about 2 days after administration, about 1 week after administration, about 2 weeks after administration, about 3 weeks after administration, about 1 month after administration, about 3 months after administration, about 6 months after administration, or about 1 year after administration.

Briefly, a psychological support session refers to a period of time where a subject participates in psychotherapy or other psychological support treatment with one or more mental health professionals and/or non-licensed facilitators. The psychological support sessions can include individual sessions, wherein a single subject meets with a single or multiple mental health professional(s). Mental health professional can include psychologists, psychiatrists, counselors, therapists, clinicians, social workers, doctors, nurses, and the like. The psychological support sessions can also include group sessions, wherein more than one subject meets with a single or multiple mental health professionals. Psychological support can be provided in person or remotely (e.g., via voice calling, over text-based communication such as email, or via a video calling or conferencing service). In one aspect, a combination of in-person psychological support and remote psychological support can be used. Types of psychological support can include mindfulness-based therapy, Cognitive Therapy, Cognitive Behavioral Therapy (CBT), Motivational Interviewing (MI), Motivational Enhancement Therapy (MET), Dialectical Behavior Therapy (DBT), Acceptance and Commitment Therapy (ACT), Contingency Management (CM), psychoanalytic therapy, psychodynamic therapy, transpersonal therapy, transdiagnostic therapy (e.g., method of levels therapy), eclectic therapeutic approaches, or a combination thereof. In addition to or separately from the listed types of psychological support, a mental health professional can also establish rapport with a subject, establish a strong therapeutic alliance with a subject, or a combination thereof. Furthermore, the environment and/or conditions in which the psychological support sessions take place can be structured or chosen to aid treatment. For example, a mental health professional or other suitable professional can provide therapeutic touch for a subject. As another example, an environment can be chosen or structured to elicit certain feelings (e.g., through the use of physical means, such as artwork, furnishings, and floral arrangements; auditory means, such as music and broadband noise; and olfactory means, such as aromatherapy). The environment can be chosen or structured to be calm; relaxing; to encourage or elicit introspection, insight, wonder, awe, or amazement; and/or a combination thereof.

### F. KITS

In a further aspect, the present disclosure relates to kits comprising at least one disclosed compound (e.g., psilocybin), a pharmaceutically acceptable salt, a hydrate, a solvate, or a polymorph thereof; or a pharmaceutical composition thereof and at least instructions for treating a CUD. The kit can, optionally, further comprise one or more agents known to treat the CUD.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions. In one aspect, the instructions of the kit can comprise instructions for providing psychological support as disclosed herein, such as instructions for providing the psychological support prior to administering the therapeutically effective amount of psilocybin or the pharmaceutical composition thereof, post-administration of the therapeutically effective amount of psilocybin or the pharmaceutical composition thereof, or a combination thereof.

### G. ASPECTS

The following listing of exemplary aspects supports and is supported by the disclosure provided herein.

Aspect 1. A method for the treatment of cocaine use disorder in a mammal comprising the step of administering to the mammal a therapeutically effective amount of psilocybin or a pharmaceutical composition thereof.

Aspect 2. The method of aspect 1, wherein the therapeutically effective amount of psilocybin comprises a therapeutically effective amount of a pharmaceutically acceptable salt, a hydrate, a solvate, or a polymorph of psilocybin.

Aspect 3. The method of aspect 1 or aspect 2, wherein the pharmaceutical composition comprises a pharmaceutically acceptable carrier.

Aspect 4. The method of any one of aspects 1-3, wherein the pharmaceutical composition comprises a pharmaceutically acceptable excipient.

Aspect 5. The method of any one of aspects 1-4, wherein the mammal is a human.

Aspect 6. The method of any one of aspects 1-5, wherein the mammal has been diagnosed with a need for treatment of the disorder prior to the administering step.

Aspect 7. The method of any one of aspects 1-6, further comprising the step of identifying a mammal in need of treatment of the disorder.

Aspect 8. The method of any one of aspects 1-7, wherein the therapeutically effective amount of psilocybin or a pharmaceutical composition thereof is in an oral dosage form

Aspect 9. The method of any one of aspects 1-8, further comprising providing the mammal with psychological support.

Aspect 10. The method of aspect, wherein the psychological support comprises having the mammal participate in at least one psychological support session prior to administration of the therapeutically effective amount of psilocybin or the pharmaceutical composition thereof.

Aspect 11. The method of aspect or aspect, wherein the psychological support comprises having the mammal participate in at least one psychological support session post-administration of the therapeutically effective amount of psilocybin or the pharmaceutical composition thereof.

Aspect 12. Use of psilocybin or a pharmaceutically acceptable salt, a hydrate, a solvate, or a polymorph thereof in the manufacture of a medicament for the treatment of cocaine use disorder in a mammal.

Aspect 13. The use of aspect, wherein the medicament is in an oral dosage form.

Aspect 14. A method for the treatment of cocaine use disorder in a mammal comprising: a) administering to the mammal a therapeutically effective amount of psilocybin or a pharmaceutical composition thereof; and b) providing the mammal with psychological support.

Aspect 15. The method of aspect, wherein the psychological support comprises having the mammal participate in at least one psychological support session prior to administration of the therapeutically effective amount of psilocybin or the pharmaceutical composition thereof.

Aspect 16. The method of aspect or aspect 15, wherein the psychological support comprises having the mammal participate in at least one psychological support session post-administration of the therapeutically effective amount of psilocybin or the pharmaceutical composition thereof.

Aspect 17. The method of any one of aspects-16, wherein the therapeutically effective amount of psilocybin comprises a therapeutically effective amount of a pharmaceutically acceptable salt, a hydrate, a solvate, or a polymorph of psilocybin.

Aspect 18. The method of any one of aspects-17, wherein the pharmaceutical composition comprises a pharmaceutically acceptable carrier.

Aspect 19. The method of any one of aspects any one of aspects-18, wherein the pharmaceutical composition comprises a pharmaceutically acceptable excipient.

Aspect 20. The method of any one of aspects-19, wherein the mammal is a human.

Aspect 21. The method of any one of aspects-20, wherein the mammal has been diagnosed with a need for treatment of the disorder prior to the administering step.

Aspect 22. The method of any one of aspects-21, further comprising the step of identifying a mammal in need of treatment of the disorder.

Aspect 23. The method of any one of aspects-22, wherein the therapeutically effective amount of psilocybin or a pharmaceutical composition thereof is in an oral dosage form.

Aspect 24. A kit comprising a therapeutically effective amount of psilocybin or a pharmaceutical composition thereof and at least instructions for treating a cocaine use disorder, comprising instructions for providing psychological support.

Aspect 25. The kit of aspect, wherein the therapeutically effective amount of psilocybin comprises a therapeutically effective amount of a pharmaceutically acceptable salt, a hydrate, a solvate, or a polymorph of psilocybin.

Aspect 26. The kit of aspect or aspect 25, wherein the pharmaceutical composition comprises a pharmaceutically acceptable carrier.

Aspect 27. The kit of any one of aspects-26, wherein the pharmaceutical composition comprises a pharmaceutically acceptable excipient.

Aspect 28. The kit of any one of aspects-27, further comprising a plurality of dosage forms, the plurality comprising one or more doses; wherein each dose comprises a therapeutically effective amount of psilocybin or the pharmaceutical composition thereof.

Aspect 29. The kit of any one of aspects-28, wherein the instructions comprise instructions for providing psychological support.

Aspect 30. The kit of aspect, wherein the instructions provide that the psychological support is provided prior to administering the therapeutically effective amount of psilocybin or the pharmaceutical composition thereof, post-administration of the therapeutically effective amount of psilocybin or the pharmaceutical composition thereof, or a combination thereof.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### H. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in °C or is at ambient temperature, and pressure is at or near atmospheric.

1. Results from Psilocybin-Assisted Psychotherapy in the Treatment of Cocaine Use Disorder

Disclosed in this Example for evaluating psilocybin in the treatment of CUD.

Design, Setting, and Participants. Randomized, quadruple-blind, placebo-controlled clinical trial at a major medical research center in the Deep South of the US. Participants were individuals with cocaine use disorder who were motivated to quit and without significant comorbidities, recruited between May 2015 and August 2023 with data collection completed in May 2024.

Interventions. Participants were randomized (1:1) to receive a single oral dose of psilocybin (25 mg per 70 kg of body weight) or active placebo (100 mg diphenhydramine). All participants received manualized psychotherapy that incorporated cognitive-behavioral treatment approximately 1 month before and 1 month after an all-day investigational drug treatment session.

Main Outcomes and Measures. Percentage of cocaine abstinent days, rates of complete cocaine abstinence, and time to first cocaine lapse through 180 days after end of treatment, assessed by timeline followback interview and confirmed with urinalysis. Hypotheses were formulated before data collection and analyses followed intention-to-treat principles.

Results Of the 40 participants, 33 (82.5%) were men, the median (IQR) age was 50.0 (43.8-56.0) years, 33 (82.5%) were Black, and 7 (17.5%) were White. Most participants had lower socioeconomic status, with 26 participants (65%) having an annual income of $20 000 or less. Four participants were lost to follow-up, resulting in 36 participants who completed assessments through 180 days after end of treatment. Psilocybin recipients had a higher percentage of cocaine abstinent days (β=28.95; 95% Cl, 18.22-39.67; P<0.001), greater likelihood of complete cocaine abstinence (odds ratio, 18.37; 95% Cl, 1.92-2468.17; P=007), and a reduced risk of cocaine lapse over time (hazard ratio, 0.28; 95% Cl, 0.13-0.60; P=001) than active placebo recipients. No serious adverse events occurred.

Conclusions and Relevance In this randomized clinical trial, psilocybin appeared to be safe and efficacious for treating cocaine use disorder among individuals from underrepresented and vulnerable populations.

Introduction. Global cocaine use reached record highs in 2023, with increases during the COVID-19 pandemic and an estimated 25 million people who used cocaine in the past year.¹ Cocaine is more likely to elicit dependence than other substances, and cocaine-related fatal overdoses in the US rose sharply after 2019 before declining in 2024.^{2,3} Whereas pharmacotherapies have been developed for several substance use disorders,⁴⁻⁷ medications for stimulant use disorders remain elusive. Although numerous pharmacotherapy candidates have been targeted,^{8,9} only contingency management, a behavioral intervention, is associated with increased abstinence^{10,11} alongside a reduction in mortality.¹²

Psychedelics have shown efficacy treating numerous mental health conditions.¹³⁻¹⁷ In the 1960s through the early 1970s, lysergic acid diethylamide showed promise in treating substance use disorders.^{18,19} In a recent trial of psilocybin for alcohol use disorder (AUD), the percentage of heavy drinking days was lower in the psilocybin group compared with the placebo group.²⁰ A subsequent trial of psilocybin for AUD and comorbid depression similarly found greater reductions in drinking with psilocybin compared with a low-dose control.²¹ However, another trial randomized those with AUD to psilocybin or placebo following detoxification and found no differences between groups. Brain imaging during drug sessions may have interfered with therapeutic response.²² Finally, in a study of psilocybin or nicotine patch for smoking cessation, prolonged abstinence rates were 40.5% for the psilocybin group at the 6-month follow-up vs 10% for the nicotine patch group.²³ Despite potential antiaddictive efficacy, no trials have evaluated psychedelics in the treatment of CUD. In the current trial, participants received manualized psychotherapy and were randomized to 1 all-day psilocybin session or 1 all-day active placebo session.

Methods—Study Design. This study was conducted at the University of Alabama at Birmingham (UAB), a major medical research center in the Deep South of the US, using a randomized, quadruple-blind, active placebo-controlled, parallel-group design. It was approved by the UAB institutional review board and participants provided written informed consent. No patients or members of the public were involved in the study design or conduct. This report follows the Consolidated Standards of Reporting Trials (CONSORT) reporting guideline for randomized trials.²⁴

Methods—Participants. Participants were recruited through media advertisements describing the study and seeking individuals who “use cocaine and want to quit.” Chain referral was used to recruit 2 participants. Participants self-reported sociodemographic characteristics including gender, race, and ethnicity.

Eligible participants were 25 years of age or older with a Diagnostic and Statistical Manual of Mental Disorders (Fourth Edition) (DSM-IV) diagnosis of cocaine dependence who reported use of cocaine on at least 4 separate days in the past month and a desire to cease cocaine use as indicated by a goal of complete abstinence on the Thoughts about Abstinence Questionnaire.²⁵ Additional inclusion criteria included a score of at least 3 on the Severity of Dependence Scale,²⁶ no hallucinogen use in the past 3 years, and good general health as assessed by medical history interview and physical examination. Exclusion criteria included pregnancy or breastfeeding, current psychiatric disorders other than substance abuse or nicotine dependence, current hypertension, current use of certain psychotropic medications (e.g., antidepressants, antipsychotics, and mood stabilizers), and personal or family history of psychotic or bipolar disorders.

Methods—Randomization and Masking. Participants were randomized in a 1:1 ratio by UAB's Investigational Drug Service within 48 hours of the all-day session to receive either a single oral dose (25 mg per 70 kg of body weight) of psilocybin (median [range] dose, 32 [21.6-61.6] mg) or 100 mg diphenhydramine using a random number table. Allocation concealment was maintained without further study involvement by the UAB Investigational Drug Service.

Participants, therapists, outcome assessors, and independent statisticians were masked to group assignment. Study medications were administered in identical opaque capsules. Participant expectancies for the treatment they were receiving were assessed with the Credibility/Expectancy Questionnaire (CEQ²⁷) after the final preparation session, before the first post-randomization integration session, and after the final integration session. Blinding integrity was assessed by asking participants to guess whether they received psilocybin or diphenhydramine and to rate how confident they were in their guess (0, not at all; 1, a little; 2, moderate; or 3, very much) before the first integration session; both the primary and secondary therapists were similarly asked to guess whether participants received psilocybin or diphenhydramine then rate how confident they were in their guess after the final integration session.

Methods—Procedures. Following telephone screening, participants completed 2 prescreening visits. Informed consent was obtained at the first visit, after which participants completed sociodemographic measures and an interview-administered retrospective calendar-based assessment using the timeline followback (TLFB²⁸) technique that queried cocaine and other drug use. Cocaine use was biochemically verified with urine samples testing for the presence of benzoylecgonine 300 ng/ml or greater, with a detection window of 24 to 240 hours after use.²⁹ The same retrospective calendar-based assessment using the TLFB technique was interview-administered at all subsequent appointments and queried cocaine and other drug use since the last study visit, with collection of urine samples to biochemically verify cocaine abstinence at all study visits.

Following completion of the 2 prescreening visits, participants completed a medical visit that included physical examination by a physician and a structured diagnostic interview for DSM-IV disorders³⁰ by trained staff. Qualified participants then completed 4 to 5 preparation psychotherapy sessions of approximately 2 hours each scheduled to occur once weekly, 1 all-day drug session preceded by 7 days of cocaine abstinence verified by urinalysis, 5 post randomization integration psychotherapy sessions of approximately 1 hour each scheduled to occur once weekly and with the first integration session occurring within 48 hours after the all-day drug session, and follow-up assessments 90 days (day 90) and 180 days (day 180) after the final integration session.

Psychotherapy was manualized by P.S.H. and integrated a client-centered approach used in prior psilocybin trials²² with a cognitive-behavioral treatment for CUD.^{31,32} P.S.H., a clinical psychologist, served as the primary therapist. To develop proficiency with psilocybin, he reviewed the literature and consulted with experienced colleagues. Secondary therapists were supervised by P.S.H. and included a marriage and family therapist (S.N.L.), clinical psychology doctoral students (D.H.G. and C.E.O.), and psychiatry residents

Methods—Outcomes. The first primary outcome was urinalysis-verified percentage of cocaine abstinent days measured across 6 time periods: in the past 90 days at baseline; during the prescreening period (i.e., baseline through the first preparation session); during the preparation period; during the integration period; from the final integration session through day 90; and from day 90 through day 180. The second primary outcome was complete cocaine abstinence, a dichotomous outcome defined as no use of cocaine whatsoever after the all-day drug session through day 180 verified by urinalysis and with participants lost to follow-up coded as non-abstinent. The final primary outcome was time to cocaine lapse, measured as days until first reported cocaine use since the all-day drug session.

Participants were observed throughout the all-day drug session and specifically queried for any drug-related adverse events (AEs) at the outset of the first integration session. Any new or worsening AEs observed or reported during or after the all-day drug session were deemed related to the intervention based on temporal proximity, participant attribution, and investigators' judgment; AEs were deemed expected from the intervention based on literature review and investigators' judgment.

Methods—Statistical Analysis. No power calculations were conducted before study initiation. As determined a priori, groups were tested on the first 2 primary outcomes before randomization to confirm group comparability and detect potential sources of bias, ensuring valid causal inference.³³ No multiplicity adjustments were applied, consistent with recommendations that they can lead to more interpretive errors than transparent reporting without adjustment.^{34,35} Unadjusted 2-sided P values of 0.05 or lower were considered statistically significant. P values are reported to the nearest hundredth unless they are less than 0.01, in which case they are reported to the nearest thousandth, allowing for readers to apply their preferred multiplicity corrections.

Mixed models for repeated measures (MMRMs) using first-order autoregressive covariance structures compared groups on percentage of cocaine abstinent days while accounting for longitudinal trends, within-participant variability, and missing data.³⁶ An MMRM first compared groups across pre-randomization time periods. An MMRM then compared groups across post-randomization time periods. This model was fitted with a group-by-time period interaction term for pairwise comparisons at each post-randomization time period. Estimated marginal means were extracted from this model, from which contrasts and standardized effect sizes (Hedges g) were calculated.

Fisher exact test and Firth penalized logistic regression³⁷ first compared groups on complete cocaine abstinence from the prescreening period through the preparation period. Fisher exact test and Firth penalized logistic regression were then used to compare groups on complete cocaine abstinence from the integration period through day 180.

Kaplan-Meier survival curves were plotted to show time to cocaine lapse by group, with a log-rank test performed for initial univariate comparison. A Cox proportional hazards model was then used to calculate a hazard ratio, assuming noninformative censoring.

Analyses included all randomized participants per intention-to-treat and were conducted using R version 2024.04.02 (R Project for Statistical Computing). This trial was monitored by an independent data monitoring committee and prospectively registered at ClinicalTrials.gov, where it was first posted on Jan. 15, 2014, before the first participant was enrolled.

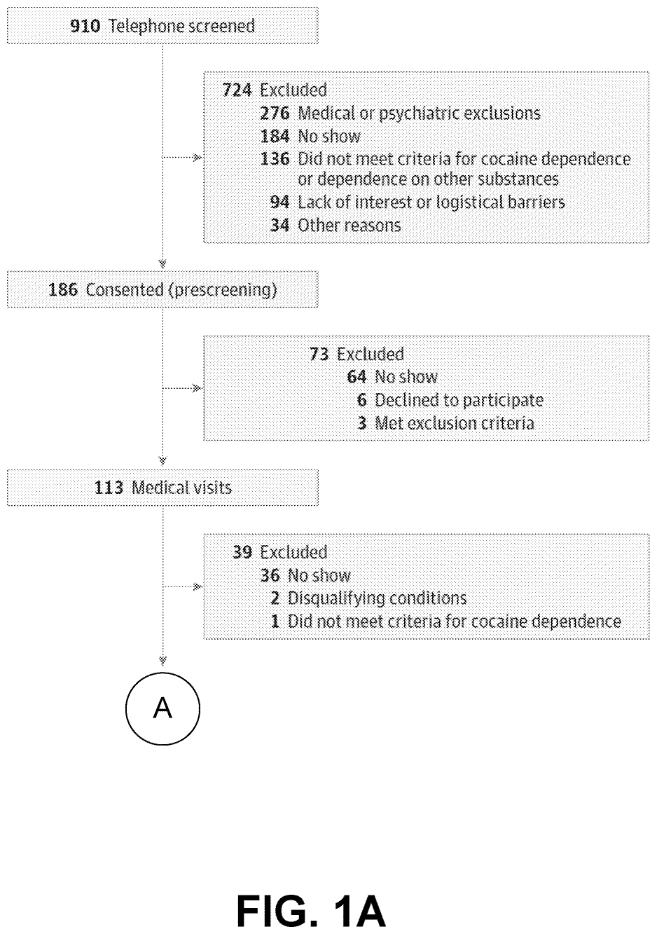

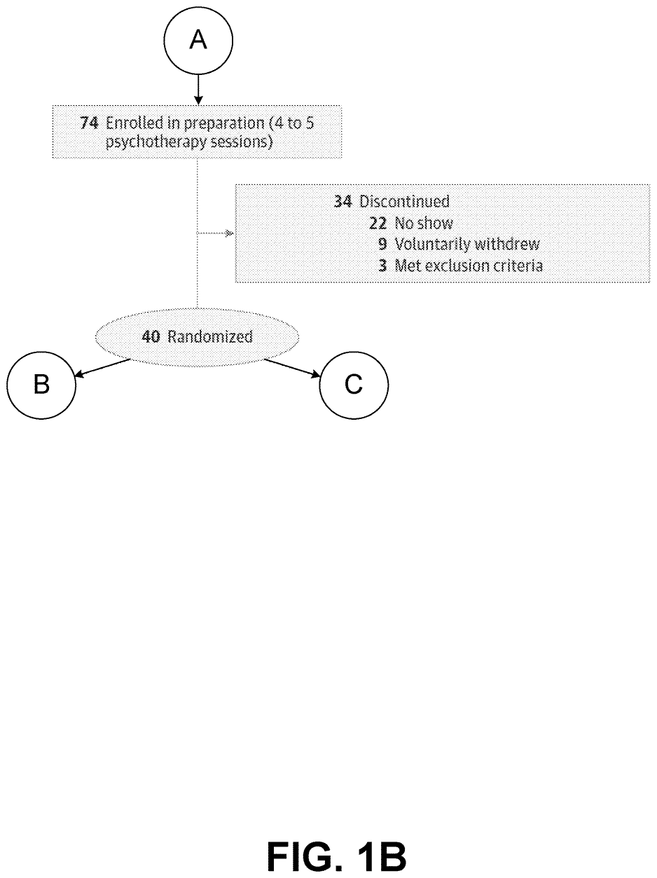

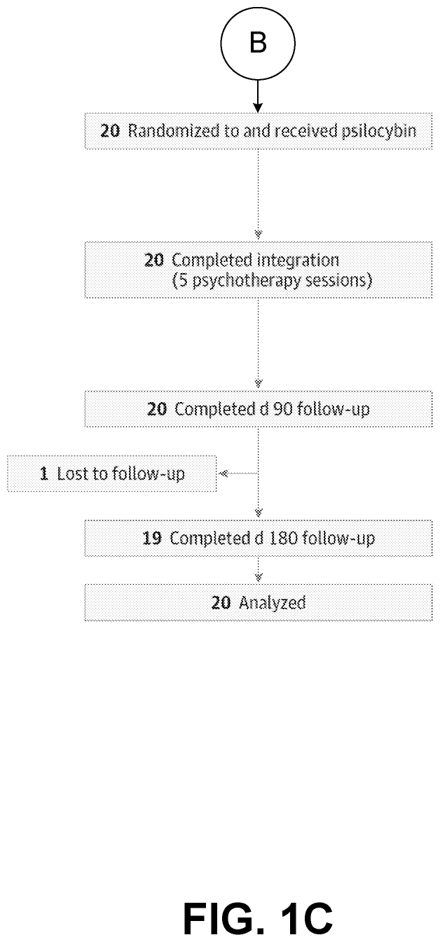

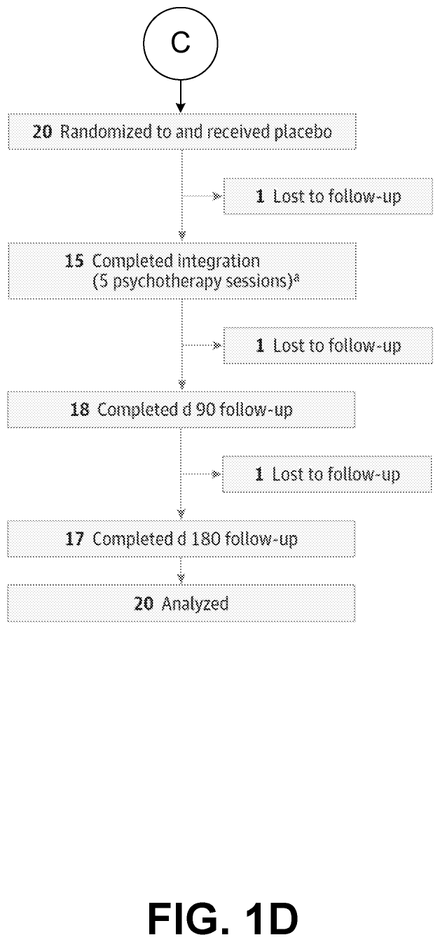

Results—Participants. Participants were recruited between May 26, 2015, and Aug. 8, 2023, and data collection for all participants was completed on May 7, 2024. Nine hundred and ten participants were assessed for eligibility, and 186 consented. Of these, 74 qualified for participation, and 40 were randomized. Four participants were lost to follow-up, leaving 36 who completed assessments through day 180 (FIGS. 1A-1D). Enrollment and data collection spanned 9 years due to budget constraints, sociopolitical considerations, and the COVID-19 pandemic. The number of days from the first participant's drug administration date to each subsequent participant's drug administration date did not differ between groups (Wilcoxon rank sum test [W], 202.00; P=0.97; standardized mean difference [SMD], 0.004), indicating psilocybin and placebo participants were equally distributed across the data collection period.

Baseline sociodemographic (Table 1) and drug use history characteristics (Table 2) of the intention-to-treat population were similar between groups. Thirty-three participants (82.5%) were men, the median (IQR) age was 50.0 (43.8-56.0) years, 33 (82.5%) were Black, and 7 (17.5%) were White. Most participants had lower socioeconomic status, with 26 participants (65%) having an annual income of $20 000 or less. No participant identified as Hispanic or Latino. Most participants smoked cocaine, with cocaine use spanning more than 2 decades. Approximately half of the sample reported daily tobacco use, heavy drinking, and cannabis use in the past 90 days. Three participants reported prior hallucinogen use, 2 of whom reported 1-time experimental use in the 1970s.

TABLE 1
Baseline Sociodemographic Characteristics of the Intention-to-Treat Population.

| | Participants, No. (%) | | |
|---|---|---|---|
| | Psilocybin | Placebo | |
| Characteristic | N = 20 | N = 20 | p-value |
| Age, median (IQR), y | 49 (43.0-55.0) | 52 (44.5-58.5) | 0.4 |
| Gender | | | 0.091 |
| Male | 14 (70.0%) | 19 (95.0%) | |
| Female | 6 (30.0) | 1 (5.0) | |
| Race/Ethnicity | | | >0.9 |
| White | 4 (20.0) | 3 (15.0) | |
| Black | 16 (80.0) | 17 (85.0) | |
| Sexual Identity | | | 0.5 |
| Heterosexual | 17 (85.0) | 14 (70.0) | |
| Sexual Minority | 3 (15.0) | 6 (30.0) | |
| Marital Status | | | 0.3 |
| Married | 5 (25.0) | 2 (10.0) | |
| Widowed | 1 (5.0) | 4 (20.0) | |
| Divorced or separated | 5 (25.0) | 7 (35.0) | |
| Single, never married | 9 (45.0) | 7 (35.0) | |
| Annual Individual Income | | | >0.9 |
| < $10,000 | 9 (45.0) | 8 (40.0) | |
| $11,000-$20,000 | 4 (20.0) | 5 (25.0) | |
| $21,000-$30,000 | 1 (5.0) | 2 (10.0) | |
| $31,000-$40,000 | 2 (10.0) | 2 (10.0) | |
| $41,000-$50,000 | 1 (5.0) | 1 (5.0) | |
| $51,000-$60,000 | 2 (10.0) | 1 (5.0) | |
| $61,000-$70,000 | 1 (5.0) | 0 (0.0) | |
| $91,000-$100,000 | 0 (0.0) | 1 (5.0) | |
| Previous Psychedelic Use | 2 (10.0) | 1 (5.0) | >0.9 |
| Employment Status | | | 0.9 |
| Employed | 9 (45.0) | 7 (35.0) | |
| Unemployed | 8 (40.0) | 9 (45.0) | |
| Retired or disabled | 3 (15.0) | 2 (10.0) | |
| Homemaker | 0 (0.0) | 1 (5.0) | |
| Student | 0 (0.0) | 1 (5.0) | |
| Veteran Status | 3 (15.0) | 3 (15.0) | >0.9 |
| Living Situation | | | 0.11 |
| Housed | 15 (75.0) | 19 (95.0) | |
| Therapeutic Housing | 1 (5.0) | 1 (5.0) | |
| Experiencing Homeless | 4 (20.0) | 0 (0.0) | |
| Education Level | | | 0.7 |
| Less than High School | 4 (20.0) | 5 (25.0) | |
| High School / GED | 8 (40.0) | 9 (45.0) | |
| Some College / AA | 6 (30.0) | 3 (15.0) | |
| Bachelor's Degree | 2 (10.0) | 3 (15.0) | |

TABLE 2
Baseline Drug Use History Characteristics of the Intention-to-Treat Population.

| | Participants, No. (%) | |
|---|---|---|
| | Psilocybin | Placebo |
| Characteristic | N = 20 | N = 20 |
| Preferred route of cocaine administration | | |
| Smoking | 13 (65.0) | 14 (70.0) |
| Snorting | 7 (35.0) | 5 (25.0) |
| Both smoking and snorting | 0 | 1 (5.0) |
| Age at first cocaine use, median (IQR), y | 22 (19.0-26.0) | 22 (18.0-25.0) |
| Age of onset of regular cocaine use, | 26 (23.5-29.0) | 26 (23.0-31.5) |
| median (IQR) | | |
| No. of prior cocaine quit attempts, median | 3 (2.0-6.0) | 4.5 (2.5-8.5) |
| (IQR) | | |
| Lifetime history of treatment for cocaine | 13 (65.0) | 16 (80.0) |
| use | | |
| Longest duration of cocaine abstinence, | 458 (106.5-1095.0) | 333 (124.0-1277.5) |
| median (IQR), d | | |
| Daily tobacco use in the past 90 d | 7 (35.0) | 11 (55.0) |
| Any heavy drinking in the past 90 d^{a} | 13 (65.0) | 11 (55.0) |
| Heavy drinking days among those | 16.7 (6.7-31.1) | 25.6 (4.4-62.8) |
| reporting any heavy drinking, median | | |
| (IQR), % | | |
| Any cannabis use in the past 90 d | 10 (50.0) | 9 (45.0) |
| Cannabis use days among those reporting | 51.7 (5.8-100.0) | 47.8 (22.2-100.0) |
| any cannabis use, median (IQR), % | | |
| Any nonmedical depressant use in the past | 1 (5.0) | 2 (10.0) |
| 90 d | | |
| Any methamphetamine or nonmedical | 2 (10.0) | 1 (5.0) |
| stimulant use in the past 90 d | | |
| Any nonmedical opioid use in the past 90 d | 1 (5.0) | 1 (5.0) |
| Any heroin use in the past 90 d | 0 | 1 (5.0) |
| Lifetime history of hallucinogen use | 2 (10.0) | 1 (5.0) |

^{a}Heavy drinking means 4 or more standard drinks on any single day for women and 5 or more standard drinks on any single day for men.

Results—Blinding Integrity. Although psilocybin and placebo participants reported similar mean [SD] CEQ credibility scores after the final preparation session (psilocybin, 9.02 [1.54]; placebo, 8.98 [1.69]; W, 180.00; P≥0.99), psilocybin participants reported greater mean (SD) CEQ credibility scores before the first integration session (psilocybin, 9.50 [1.01]; placebo, 9.07 [0.87]; W, 259.50; P=0.03; rank-biserial r=0.18), with group differences between mean (SD) scores not significant after the final integration session (psilocybin, 9.95 [0.18]; placebo, 9.56 [0.83]; W, 120.00; P=0.05; rank-biserial r=0.16). Although mean (SD) CEQ expectancy scores were similar between groups after the final preparation session (psilocybin, 9.35 [1.21]; placebo, 9.17 [0.96]; W, 216.50; P=0.26), psilocybin participants reported greater mean (SD) CEQ expectancy scores before the first integration session (psilocybin, 9.70 [0.52]; placebo, 8.68 [1.11]; W, 298.50; P=0.001; rank-biserial r=0.29) and after the final integration session (psilocybin, 9.90 [0.20]; placebo, 8.62 [1.50]; W, 131.00; P=0.02; rank-biserial r=0.22).

Eighteen of 20 psilocybin participants (90%) correctly guessed their allocation (mean [SD] confidence score, 2.90 [0.44]), compared with 9 of 19 placebo participants (47%; mean [SD] confidence score, 2.26 [0.65]). The primary therapist and secondary therapists each correctly identified allocation in 39 of 40 cases (97.5%) with mean (SD) confidence scores of 2.65 (0.58) and 2.80 (0.41) when guessing allocation among participants who received psilocybin and 2.89 (0.31) and 2.63 (0.59) when guessing allocation among those who received diphenhydramine, respectively.

Results—Treatment Process. Participants in the psilocybin group and placebo group spent an equivalent mean (SD) number of days in the prescreening (psilocybin, 26.85 [13.75]; placebo, 28.65 [21.38]; W, 203.50; P=0.93) and preparation (psilocybin, 33.65 [20.86]; placebo, 29.90 [10.64]; W, 212.50; P=0.74) periods. Four psilocybin participants and 3 placebo participants completed a fifth preparation session to address nonadherence to the study protocol. Psilocybin participants spent more mean (SD) days in the integration period than placebo participants (psilocybin, 64.25 [44.18]; placebo, 41.42 [26.19]; W, 112.00; P=0.02; SMD, 0.62); whereas all psilocybin participants completed integration psychotherapy, only 15 of 20 placebo participants (75%) did the same. Of the 5 placebo participants who did not complete integration psychotherapy, 1 attended no integration sessions, 1 completed 1 integration session, 1 completed 2 integration sessions, and 2 completed 4 integration sessions.

Results—Biological Verification of Abstinence. Urine samples were labeled discordant if positive for the presence of benzoylecgonine 300 ng/ml or more despite no self-reported cocaine use in the past 7 days. Three participants yielded discordant urine samples: 1 psilocybin participant at the second prescreening visit; 1 placebo participant at the second prescreening visit; and 1 placebo participant at the second prescreening visit and fourth preparation session. In each instance, participants were reminded that cocaine use would not be penalized and the priority of the study was to obtain accurate data. They were given the opportunity to modify their self-report but chose not to do so, and their data were entered as reported. None of these participants reported complete abstinence in the prescreening or preparation periods and no further discordant urine samples were obtained.

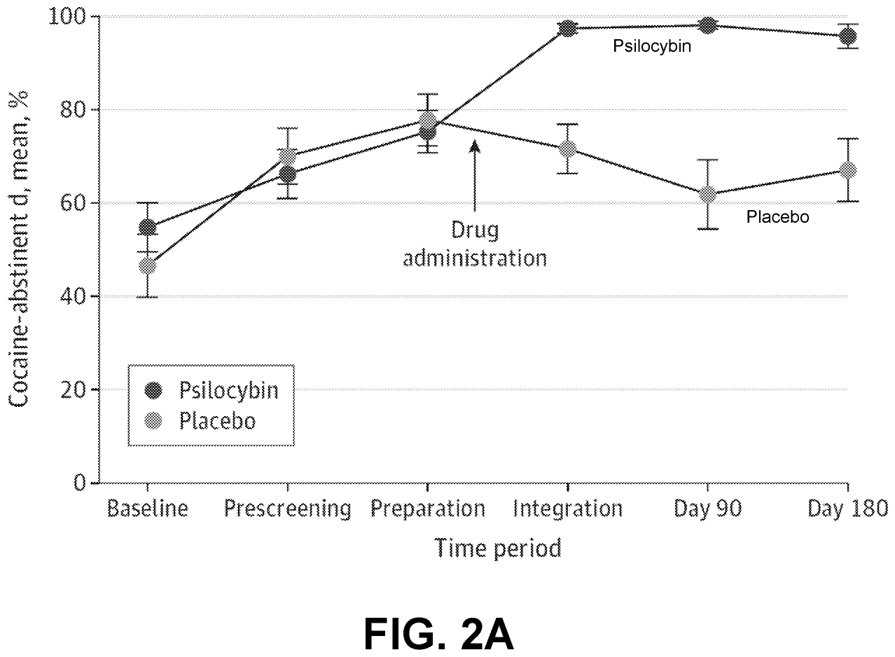

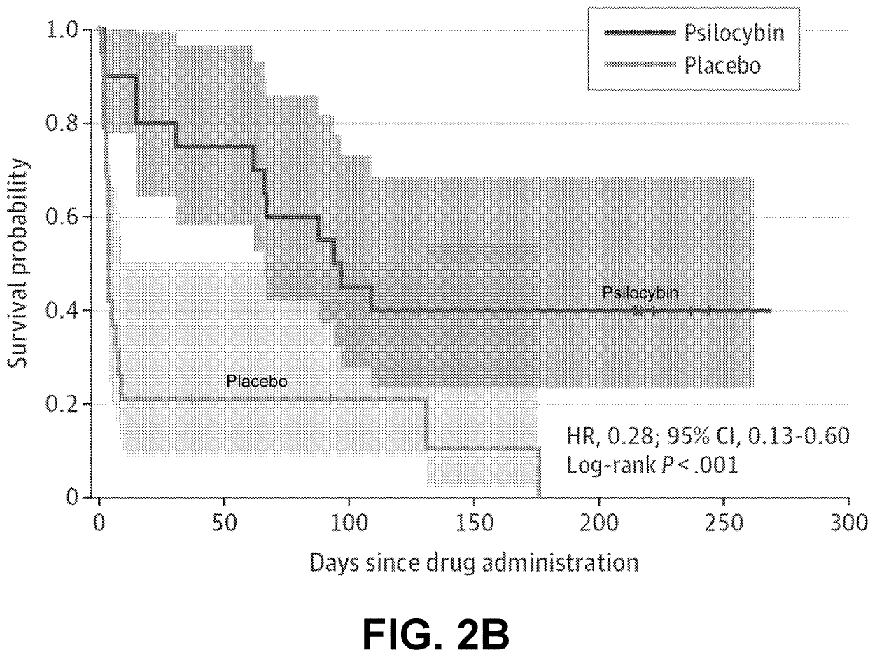

Results—Outcomes. FIG. 2A presents the mean percentage of cocaine abstinent days across the study by group. An MMRM comparing groups across pre-randomization time periods did not reveal a main effect of group (β=0.94; 95% Cl, −14.01 to 15.90; P=0.89), although main effects of prescreening (β=16.22; 95% Cl, 9.91 to 22.53; P<001) and preparation (β=24.54; 95% Cl, 16.37 to 32.71; P<0.001) periods were observed, indicating that participants in both groups reported an increase in abstinence from baseline before drug administration. An MMRM comparing groups across post-randomization time periods demonstrated a main effect (β=28.95; 95% Cl, 18.22 to 39.67; P<0.001), with participants in the psilocybin group reporting more abstinence than participants in the placebo group. No significant group-by-time period interactions were observed, suggesting a stable treatment effect over time. Pairwise comparisons indicated that psilocybin participants reported 26.31 (Hedges g=1.40; 95% Cl, 0.73 to 2.08; P<0.001), 35.88 (Hedges g=1.92; 95% Cl, 1.23 to 2.60; P<0.001), and 28.36 (Hedges g=1.51; 95% Cl, 0.82 to 2.21; P<0.001) more percentage points of abstinent days than the placebo participants during the integration period, from the final integration session through day 90 and from day 90 through day 180, respectively. There was no indication that psilocybin participants substituted methamphetamine, nonmedical stimulants, or other drugs for cocaine (these results will be reported in a separate publication).

No participants reported complete abstinence during the prescreening period, whereas 2 of 20 psilocybin participants (10%) and 3 of 20 placebo participants (15%) reported complete abstinence during the preparation period; Fisher exact test (P>0.99) and Firth penalized logistic regression (odds ratio [OR], 0.67; 95% Cl, 0.10-3.91; P=0.65) failed to reveal an association between group and complete abstinence during this time. None of these 5 participants reported complete abstinence from the integration period through day 180. Six of 20 psilocybin participants (30%) and 0 of 20 placebo participants reported complete abstinence from the integration period through day 180; Fisher exact test (P=0.02) showed a significant association between group and complete abstinence, with Firth penalized logistic regression indicating that psilocybin participants were approximately 18 times more likely to report complete abstinence than placebo participants (OR, 18.37; 95% Cl, 1.92-2468.17; P=0.007), and a number needed to treat of 3.33 (95% Cl, 2.0-10.1). The wide Cls reflect the small sample size and data separation in this variable-outcome combination.

Kaplan-Meier estimates (FIG. 2B) demonstrated survival probabilities of 55.0% (95% Cl, 36.99%-81.75%) and 21.05% (95% Cl, 8.81%-50.28%) at 90 days in the psilocybin and placebo groups, respectively (log-rank χ²^{₁}=11.65; P<0.001). Participants in the psilocybin group had a significantly reduced risk of lapse compared with participants in the placebo group (hazard ratio, 0.28; 95% Cl, 0.13-0.60; P=0.001).

AEs occurred in 13 of 20 psilocybin participants (65%) and 2 of 20 placebo participants (10%). No serious treatment-related AEs occurred. Most AEs were expected and occurred during the all-day drug session, and all were resolved with no sequelae. No rescue medications were administered. The most commonly reported adverse events in the psilocybin condition were transient hypertension during the all-day drug session (30%), emotional distress during the all-day drug session (25%), crying during the all-day drug session (25%), and headache following the all-day drug session (15%). Other adverse events reported in the psilocybin condition included tachycardia, flatulence, hunger, vomiting, hot flashes with chills, spontaneous orgasm, post-session agitation, post-session suicidal ideation, altered perception, and heightened visual imagery.

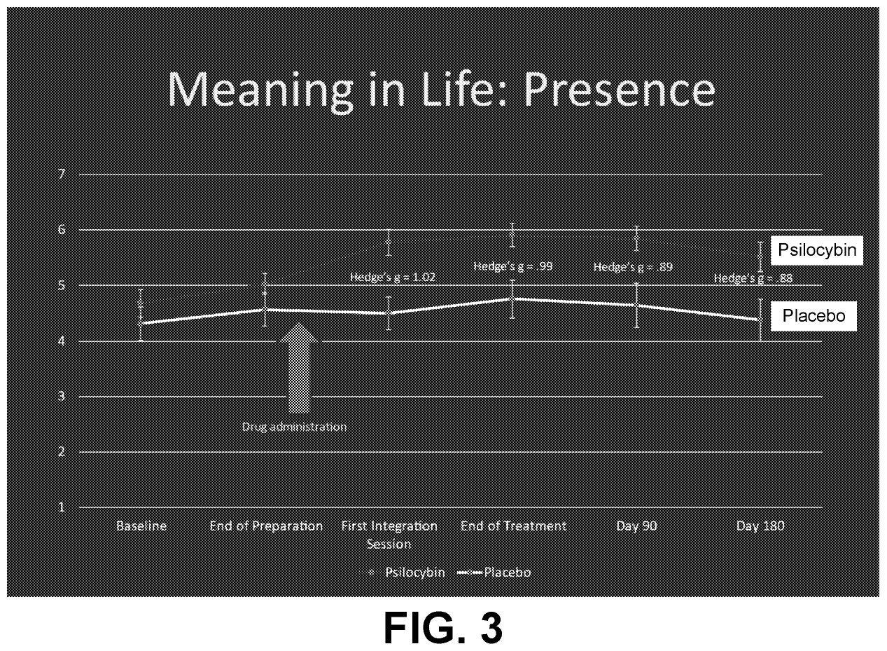

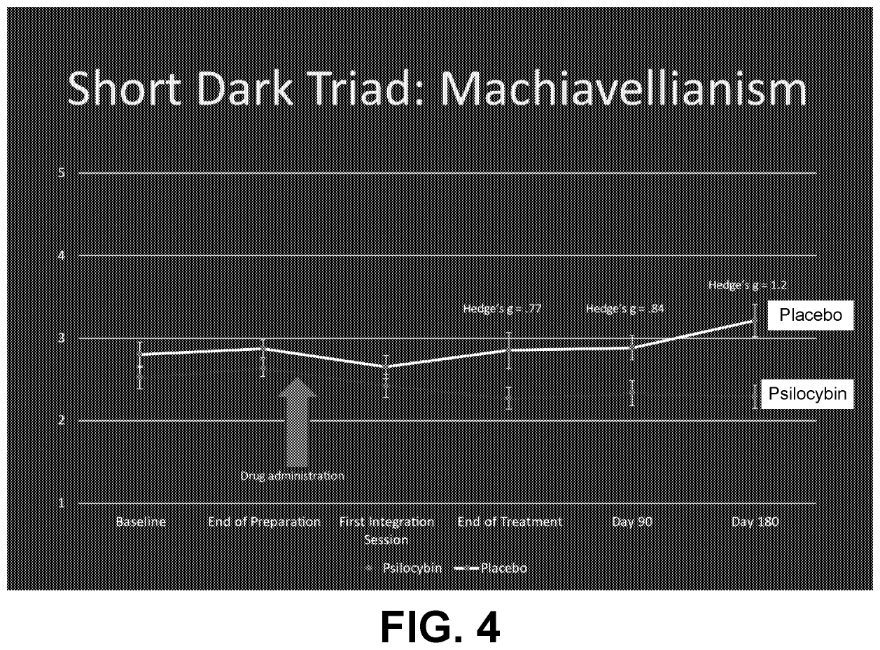

As illustrated in FIG. 3, psilocybin treatment resulted in increases to meaning in life (determined by the Meaning in Life questionnaire, Presence scale) relative to placebo treatment for individuals with CUD. As illustrated in FIG. 4, psilocybin treatment resulted in decreases to Machiavellianism (determined by the Short Dark Triad, Machiavellianism scale) relative to placebo treatment for individuals with CUD. In both figures, Hedge's g effect sizes are reported only when one-way ANOVAs revealed statistically significant differences between the conditions at p<05, two-tailed. Note that the two conditions did not differ prior to drug administration.

Secondary Outcomes. Secondary outcomes include: (1) severity of cocaine dependence, as measured by the Severity of Dependence Scale (SDS); (2) percentage of drinking and heavy drinking days, assessed via the TLFB; (3) percentage of days abstinent and complete abstinence from cannabis, assessed via the TLFB; (4) percentage of days abstinent and complete abstinence from other illicit drugs, assessed via the TLFB; (5) percentage of days abstinent and complete abstinence from tobacco, assessed via the TLFB; (6) subjective drug effects as mediators of treatment, assessed via the Mystical Experience Questionnaire (MEQ), Challenging Experience Questionnaire (CEQ), Abnormal Mental States (APZ) questionnaire, and Hood Mysticism Scale; (7) other potential mediators of treatment, including cocaine withdrawal, craving, abstinence self-efficacy, and motivation to quit/remain abstinent; (8) indicators of quality of life, including the Satisfaction with Life Scale (SLS), the Depression, Anxiety, and Stress Scale (DASS-21), and self-reported changes in income, employment status, and living situation; (9) a range of psychological constructs, including meaning in life (Meaning in Life Questionnaire), self-compassion (Short Self-Compassion Scale), Machiavellianism, narcissism, and psychopathy (Short Dark Triad), measures of prosocial attitudes and behavior (e.g., Trait Forgiveness Scale), and measures of positive emotions (e.g., Dispositional Positive Emotions Scale); and (10) neurobiological changes (using MRI/fMRI/MRS). These outcomes are measured at various intervals. See Table 3 for more information.

TABLE 3
Outcome measures.

| Type | Outcome | Measurement Method | Time Point(s) |
|---|---|---|---|
| Primary | Percentage of | Mean percentage via TLFB | Baseline, prescreen, |
| | days abstinent | verified with urine drug screens | preparation, integration, |
| | from cocaine | | Day 90, Day 180 |
| Primary | Complete | Dichotomous (abstinent or | Baseline, prescreen, |
| | abstinence | abstinent or non-abstinent; yes | preparation, integration, |
| | from cocaine | or no) via TLFB verified with | Day 90, Day 180 |
| | | urine drug screens | |
| Primary | Time to first | Days to first cocaine use after | Integration through Day |
| | cocaine lapse | drug administration via TLFB | 180 |
| | | verified with urine drug screens | |
| Secondary | Severity of | Mean SDS scores (self-report) | Beginning of preparation, |
| | cocaine | | end of preparation/MRI 1, |
| | dependence | | beginning of |
| | | | integration/MRI 2, end-of- |
| | | | treatment, Day 90, Day |
| | | | 180 |
| Secondary | Percentage of | Mean percentage via TLFB | Baseline, prescreen, |
| | drinking and | | preparation, integration, |
| | heavy drinking | | Day 90, Day 180 |
| | days | | |
| Secondary | Percentage of | Mean percentage and | Baseline, prescreen, |
| | days abstinent | dichotomous (abstinent or | preparation, integration, |
| | and complete | abstinent or non-abstinent; yes | Day 90, Day 180 |
| | abstinence | or no) via TLFB | |
| | from cannabis | | |
| Secondary | Percentage of | Mean percentage and | Baseline, prescreen, |
| | days abstinent | dichotomous (abstinent or | preparation, integration, |
| | and complete | abstinent or non-abstinent; yes | Day 90, Day 180 |
| | abstinence | or no) via TLFB | |
| | from other illicit | | |
| | substances | | |
| Secondary | Percentage of | Mean percentage and | Baseline, prescreen, |
| | days abstinent | dichotomous (abstinent or | preparation, integration, |
| | and complete | abstinent or non-abstinent; yes | Day 90, Day 180 |
| | abstinence | or no) via TLFB | |
| | from tobacco | | |
| Secondary | Subjective drug | Mean MEQ, CEQ, APZ, and | At the end of the drug |
| | effects | Hood Mysticism Scale (self- | administration day |
| | | report) scores | |
| Secondary | Cocaine | Mean CSSA (interview), CCQ- | Beginning of preparation, |
| | withdrawal, | Brief (self-report), BSCQ (self- | end of preparation/MRI 1, |
| | craving, | report), and Thoughts about | beginning of |
| | abstinence | Abstinence (self-report) scores | integration/MRI 2, end-of- |
| | self-efficacy, | | treatment, Day 90, Day |
| | motivation to | | 180 |
| | quit/remain | | |
| | abstinent | | |
| Secondary | Indicators of | Mean Satisfaction with Life | Beginning of preparation, |
| | quality of life | Scale (self-report) and DASS- | end of preparation/MRI 1, |
| | | 21 (self-report) scores; | beginning of |
| | | responses via demographic | integration/MRI 2, end-of- |
| | | questionnaire (self-report) on | treatment, Day 90, Day |
| | | changes in income, | 180; demographic |
| | | employment, and living | questionnaire at Baseline |
| | | situation | and Day 180 only |
| Secondary | Psychological | Mean scores across a number | Beginning of preparation, |
| | constructs | of self-report measures, | end of preparation/MRI 1, |
| | | including the Meaning in Life | beginning of |
| | | Questionnaire, Short Self- | integration/MRI 2, end-of- |
| | | Compassion Scale, Short Dark | treatment, Day 90, Day |
| | | Triad, Trait Forgiveness Scale, | 180 |
| | | and Dispositional Positive | |
| | | Emotions Scale | |
| Secondary | Neurobiological | MRI/fMRI/MRS | MRI 1: 2 days after final |
| | changes (e.g., | | preparation session |
| | changes to | | MRI 2: within 48 hours |
| | default mode | | after drug administration |
| | network | | |
| | functional | | |
| | connectivity) | | |

Discussion. This randomized clinical trial is understood to be the first to demonstrate that psilocybin coupled with psychotherapy may be safe and efficacious in the treatment of CUD. Psilocybin-treated participants showed significantly greater percentages of cocaine abstinent days, higher rates of complete abstinence from cocaine, and a decreased risk of cocaine lapse over time. These findings are a potentially important advancement in the treatment of CUD, a condition for which there are no approved pharmacotherapies and limited psychosocial interventions.¹¹

The representation of vulnerable populations in psychedelic clinical trials has been a ongoing concern.^{38,39} A recent systematic review found that participants in US-based psychedelic trials typically had higher socioeconomic status than the general population.³⁸ The present study demonstrates that psilocybin treatment can be feasibly implemented with Black and socioeconomically disadvantaged individuals vulnerable to the adverse impacts of CUD but understudied in psychedelic research.^{38,39}

Conclusions. In this randomized clinical trial, psilocybin appeared safe and efficacious for CUD, a difficult-to-treat condition with significant personal and public health impacts.

### I. REFERENCES

References are cited herein throughout using the format of reference number(s) superscripted corresponding to one or more of the following numbered references. For example, citation of references numbers 1 and 2 immediately herein below would be indicated in the disclosure as 1,2.
Ref 1 United Nations Office on Drugs and Crime. World Drug Report 2025. United Nations publication, 2025. Accessed Mar. 26, 2026. https://www.unodc.org/unodc/data-and-analysis/world-drug-report-2025.html
Ref 2 Lopez-Quintero C, Pérez de los Cobos J, Hasin D S, et al. Probability and predictors of transition from first use to dependence on nicotine, alcohol, cannabis, and cocaine: results of the National Epidemiologic Survey on Alcohol and Related Conditions (NESARC). Drug Alcohol Depend. 2011; 115(1-2):120-130. doi: 10.1016/j.drugalcdep.2010.11.004
Ref 3 Garnett M F, Miniño A M. Drug overdose deaths in the United States, 2023-2024. NCHS Data Brief. 2026; (549). doi: 10.15620/cdc/174639PubMedGoogle Scholar
Ref 4 Degenhardt L, Clark B, Macpherson G, et al. Buprenorphine versus methadone for the treatment of opioid dependence: a systematic review and meta-analysis of randomised and observational studies. Lancet Psychiatry. 2023; 10(6):386-402. doi: 10.1016/S2215-0366(23)00095-0
Ref 5 Lim J, Farhat I, Douros A, Panagiotoglou D. Relative effectiveness of medications for opioid-related disorders: a systematic review and network meta-analysis of randomized controlled trials. PLOS One. 2022; 17(3):e0266142. doi: 10.1371/journal.pone.0266142
Ref 6 Agabio R, Lopez-Pelayo H, Bruguera P, et al. Efficacy of medications for the treatment of alcohol use disorder (AUD): a systematic review and meta-analysis considering baseline AUD severity. Pharmacol Res. 2024; 209:107454. doi: 10.1016/j.phrs.2024.107454
Ref 7 Rigotti N A, Kruse G R, Livingstone-Banks J, Hartmann-Boyce J. Treatment of tobacco smoking: a review. JAMA. 2022; 327(6):566-577. doi: 10.1001/jama.2022.0395
Ref 8 Li M J, Chau B, Belin T, et al. Extended observation of reduced methamphetamine use with combined naltrexone plus bupropion in the ADAPT-2 trial. Addiction. 2024; 119(10):1840-1845. doi: 10.1111/add. 16529
Ref 9 Chan B, Kondo K, Freeman M, Ayers C, Montgomery J, Kansagara D. Pharmacotherapy for cocaine use disorder—a systematic review and meta-analysis. J Gen Intern Med. 2019; 34(12):2858-2873. doi: 10.1007/s11606-019-05074-8
Ref 10 Petry N M. Contingency management treatments: controversies and challenges. Addiction. 2010; 105(9):1507-1509. doi: 10.1111/j.1360-0443.2009.02879.x
Ref 11 Bentzley B S, Han S S, Neuner S, Humphreys K, Kampman K M, Halpern C H. Comparison of treatments for cocaine use disorder among adults: a systematic review and meta-analysis. JAMA Netw Open. 2021; 4(5):e218049. doi: 10.1001/jamanetworkopen.2021.8049
Ref 12 Coughlin L N, Tomlinson D C, Zhang L, et al. Contingency management for stimulant use disorder and association with mortality: a cohort study. Am J Psychiatry. 2025; 182(11):1016-1023. doi: 10.1176/appi.ajp.20250053
Ref 13 Goodwin G M, Aaronson S T, Alvarez O, et al. Single-dose psilocybin for a treatment-resistant episode of major depression. N Engl J Med. 2022; 387(18):1637-1648. doi: 10.1056/NEJMoa2206443
Ref 14 Raison C L, Sanacora G, Woolley J, et al. Single-dose psilocybin treatment for major depressive disorder: a randomized clinical trial. JAMA. 2023; 330(9):843-853. doi: 10.1001/jama.2023.14530
Ref 15 Griffiths R R, Johnson M W, Carducci M A, et al. Psilocybin produces substantial and sustained decreases in depression and anxiety in patients with life-threatening cancer: a randomized double-blind trial. J Psychopharmacol. 2016; 30(12):1181-1197. doi: 10.1177/0269881116675513
Ref 16 Ross S, Bossis A, Guss J, et al. Rapid and sustained symptom reduction following psilocybin treatment for anxiety and depression in patients with life-threatening cancer: a randomized controlled trial. J Psychopharmacol. 2016; 30(12):1165-1180. doi: 10.1177/0269881116675512
Ref 17 Anderson B T, Danforth A, Daroff P R, et al. Psilocybin-assisted group therapy for demoralized older long-term AIDS survivor men: an open-label safety and feasibility pilot study. EClinicalMedicine. 2020; 27:100538. doi: 10.1016/j.eclinm.2020.100538
Ref 18 Krebs T S, Johansen PØ. Lysergic acid diethylamide (LSD) for alcoholism: meta-analysis of randomized controlled trials. J Psychopharmacol. 2012; 26(7):994-1002. doi: 10.1177/0269881112439253
Ref 19 Savage C, McCabe O L. Residential psychedelic (LSD) therapy for the narcotic addict. A controlled study. Arch Gen Psychiatry. 1973; 28(6):808-814. doi: 10.1001/archpsyc. 1973.01750360040005
Ref 20 Bogenschutz M P, Ross S, Bhatt S, et al. Percentage of heavy drinking days following psilocybin-assisted psychotherapy vs placebo in the treatment of adult patients with alcohol use disorder: a randomized clinical trial. JAMA Psychiatry. 2022; 79(10):953-962. doi: 10.1001/jamapsychiatry.2022.2096
Ref 21 Luquiens A, Belahda D, Graux C, et al. Psilocybin in alcohol use disorder and comorbid depressive symptoms: results from a feasibility randomized clinical trial. Addiction. Published online Jul. 24, 2025. doi: 10.1111/add.70152
Ref 22 Rieser N M, Bitar R, Halm S, et al. Psilocybin-assisted therapy for relapse prevention in alcohol use disorder: a phase 2 randomized clinical trial. EClinicalMedicine. 2025; 82:103149. doi: 10.1016/j.eclinm.2025.103149
Ref 23 Johnson M W, Naude G P, Hendricks P S, Garcia-Romeu A. Psilocybin or nicotine patch for smoking cessation: a pilot randomized clinical trial. JAMA Netw Open. 2026; 9(3):e260972. doi: 10.1001/jamanetworkopen.2026.0972
Ref 24 Hopewell S, Chan A W, Collins G S, et al. CONSORT 2025 statement: updated guideline for reporting randomised trials. BMJ. 2025; 389: e081123. doi: 10.1136/bmj-2024-081123
Ref 25 Hall S M, Havassy B E, Wasserman D A. Effects of commitment to abstinence, positive moods, stress, and coping on relapse to cocaine use. J Consult Clin Psychol. 1991; 59(4):526-532. doi: 10.1037/0022-006X.59.4.526
Ref 26 Gossop M, Darke S, Griffiths P, et al. The Severity of Dependence Scale (SDS): psychometric properties of the SDS in English and Australian samples of heroin, cocaine and amphetamine users. Addiction. 1995; 90(5):607-614. doi: 10.1046/j. 1360-0443.1995.9056072.x
Ref 27 Devilly G J, Borkovec T D. Psychometric properties of the credibility/expectancy questionnaire. J Behav Ther Exp Psychiatry. 2000; 31(2):73-86. doi: 10.1016/S0005-7916(00)00012-4
Ref 28 Hersh D, Mulgrew C L, Van Kirk J, Kranzler H R. The validity of self-reported cocaine use in two groups of cocaine abusers. J Consult Clin Psychol. 1999; 67(1):37-42. doi: 10.1037/0022-006X.67.1.37
Ref 29 Holtyn A F, Knealing T W, Jarvis B P, Subramaniam S, Silverman K. Monitoring cocaine use and abstinence among cocaine users for contingency management interventions. Psychol Rec. 2017; 67(2):253-259. doi: 10.1007/s40732-017-0236-1
Ref 30 Sheehan D V, Lecrubier Y, Sheehan K H, et al. The Mini-International Neuropsychiatric Interview (M.I.N.I.): the development and validation of a structured diagnostic psychiatric interview for DSM-I V and ICD-10. J Clin Psychiatry. 1998; 59 (suppl 20):22-33.PubMedGoogle Scholar
Ref 31 Rohsenow D J, Monti P M, Martin R A, Michalec E, Abrams D B. Brief coping skills treatment for cocaine abuse: 12-month substance use outcomes. J Consult Clin Psychol. 2000; 68(3):515-520. doi: 10.1037/0022-006X.68.3.515
Ref 32 Rohsenow D J, Monti P M, Martin R A, et al. Motivational enhancement and coping skills training for cocaine abusers: effects on substance use outcomes. Addiction. 2004; 99(7):862-874. doi: 10.1111/j.1360-0443.2004.00743.x
Ref 33 Stang A, Baethge C. Imbalance p values for baseline covariates in randomized controlled trials: a last resort for the use of p values? A pro and contra debate. Clin Epidemiol. 2018; 10:531-535. doi: 10.2147/CLEP.S161508
Ref 34 Rothman K J. No adjustments are needed for multiple comparisons. Epidemiology. 1990; 1(1):43-46. doi: 10.1097/00001648-199001000-00010
Ref 35 Perneger T V. What's wrong with Bonferroni adjustments. BMJ. 1998; 316(7139):1236-1238. doi: 10.1136/bmj.316.7139.1236
Ref 36 Siddiqui O. MMRM versus M I in dealing with missing data—a comparison based on 25 NDA data sets. J Biopharm Stat. 2011; 21(3):423-436. doi: 10.1080/10543401003777995
Ref 37 Heinze G, Schemper M. A solution to the problem of separation in logistic regression. Stat Med. 2002; 21(16):2409-2419. doi: 10.1002/sim. 1047
Ref 38 Grossman D H, Madden K M, Mehtani N J, et al. A systematic review of income and education reporting in psychedelic clinical trials. Nat Ment Health. 2025; 3(5):567-574. doi: 10.1038/s44220-025-00417-3
Ref 39 Ortiz C E, Dourron H M, Sweat N W, et al. Special considerations for evaluating psilocybin-facilitated psychotherapy in vulnerable 2022; 214:109127. populations. Neuropharmacology. doi: 10.1016/j.neuropharm.2022.109127
Ref 40 Muthukumaraswamy S D, Forsyth A, Lumley T. Blinding and expectancy confounds in psychedelic randomized controlled trials. Expert Rev Clin Pharmacol. 2021; 14(9):1133-1152. doi: 10.1080/17512433.2021.1933434
Ref 41 Szigeti B, Weiss B, Rosas F E, Erritzoe D, Nutt D, Carhart-Harris R. Assessing expectancy and suggestibility in a trial of escitalopram v. psilocybin for depression. Psychol Med. 2024; 54(8):1717-1724. doi: 10.1017/S0033291723003653
Ref 42 Huneke N T M, Fusetto Veronesi G, Garner M, Baldwin D S, Cortese S. Expectancy effects, failure of blinding integrity, and placebo response in trials of treatments for psychiatric disorders: a narrative review. JAMA Psychiatry. 2025; 82(5):531-538. doi: 10.1001/jamapsychiatry.2025.0085
Ref 43 Nayak S M, Bradley M K, Kleykamp B A, et al. Control conditions in randomized trials of psychedelics: an ACTTION systematic review. J Clin Psychiatry. 2023; 84(3):22r14518.
Ref 44 Hieronymus F, López E, Werin Sjögren H, Lundberg J. Control group outcomes in trials of psilocybin, SSRIs, or esketamine for depression. JAMA Netw Open. 2025; 8(7):e2524119. doi: 10.1001/jamanetworkopen.2025.24119
Ref 45 Hsu T W, Tsai C K, Kao Y C, et al. Comparative oral monotherapy of psilocybin, lysergic acid diethylamide, 3,4-methylenedioxymethamphetamine, ayahuasca, and escitalopram for depressive symptoms: systematic review and Bayesian network meta-analysis. BMJ. 2024; 386: e078607. doi: 10.1136/bmj-2023-078607
Ref 46 Moustgaard H, Clayton G L, Jones H E, et al. Impact of blinding on estimated treatment effects in randomised clinical trials: meta-epidemiological study. BMJ. 2020; 368:16802. doi: 10.1136/bmj.16802
Ref 47 Fendel J C, Tiersch C, Sölder P, Gaab J, Schmidt S. Effects of open-label placebos across populations and outcomes: an updated systematic review and meta-analysis of randomized controlled trials. Sci Rep. 2025; 15(1):29940. doi: 10.1038/s41598-025-14895-zPubMedCrossref
Ref 48 Dragioti E, Dimoliatis I, Fountoulakis K N, Evangelou E. A systematic appraisal of allegiance effect in randomized controlled trials of psychotherapy. Ann Gen Psychiatry. 2015; 14:25. doi: 10.1186/s12991-015-0063-1
Ref 49 Bharat C, Webb P, Wilkinson Z, et al. Agreement between self-reported illicit drug use and biological samples: a systematic review and meta-analysis. Addiction. 2023; 118(9):1624-1648. doi: 10.1111/add. 16200
Ref 50 Susukida R, Crum R M, Stuart E A, Ebnesajjad C, Mojtabai R. Assessing sample representativeness in randomized controlled trials: application to the National Institute of Drug Abuse Clinical Trials Network. Addiction. 2016; 111(7):1226-1234. doi: 10.1111/add. 13327

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A method for the treatment of a cocaine use disorder in a mammal comprising the step of administering to the mammal a therapeutically effective amount of psilocybin or a pharmaceutical composition thereof.

2. The method of claim 1, wherein the therapeutically effective amount of psilocybin comprises a therapeutically effective amount of a pharmaceutically acceptable salt, a hydrate, a solvate, or a polymorph of psilocybin.

3. The method of claim 1, wherein the pharmaceutical composition comprises a pharmaceutically acceptable carrier.

4. The method of claim 1, wherein the pharmaceutical composition comprises a pharmaceutically acceptable excipient.

5. The method of claim 1, wherein the mammal has been diagnosed with a need for treatment of the cocaine use disorder prior to an administering step.

6. The method of claim 1, wherein the therapeutically effective amount of psilocybin or a pharmaceutical composition thereof is in an oral dosage form.

7. The method of claim 1, further comprising providing the mammal with psychological support.

8. The method of claim 7, wherein the psychological support comprises having the mammal participate in at least one psychological support session prior to administration of the therapeutically effective amount of psilocybin or the pharmaceutical composition thereof.

9. The method of claim 7, wherein the psychological support comprises having the mammal participate in at least one psychological support session post-administration of the therapeutically effective amount of psilocybin or the pharmaceutical composition thereof.

10. A method for the treatment of cocaine use disorder in a mammal comprising:
a) administering to the mammal a therapeutically effective amount of psilocybin or a pharmaceutical composition thereof; and
b) providing the mammal with psychological support.

11. The method of claim 10, wherein the psychological support comprises having the mammal participate in at least one psychological support session prior to administration of the therapeutically effective amount of psilocybin or the pharmaceutical composition thereof.

12. The method of claim 10, wherein the psychological support comprises having the mammal participate in at least one psychological support session post-administration of the therapeutically effective amount of psilocybin or the pharmaceutical composition thereof.

13. The method of claim 10, wherein the therapeutically effective amount of psilocybin comprises a therapeutically effective amount of a pharmaceutically acceptable salt, a hydrate, a solvate, or a polymorph of psilocybin.

14. The method of claim 10, wherein the therapeutically effective amount of psilocybin or a pharmaceutical composition thereof is in an oral dosage form.

15. A kit comprising a therapeutically effective amount of psilocybin or a pharmaceutical composition thereof and at least instructions for treating a cocaine use disorder, comprising instructions for providing psychological support.

16. The kit of claim 15, wherein the instructions comprise instructions for participation in at least one psychological support session.

17. The kit of claim 15, wherein the instructions provide that the psychological support is provided prior to administering the therapeutically effective amount of psilocybin or the pharmaceutical composition thereof, post-administration of the therapeutically effective amount of psilocybin or the pharmaceutical composition thereof, or a combination thereof.

18. The kit of claim 15, wherein the therapeutically effective amount of psilocybin comprises a therapeutically effective amount of a pharmaceutically acceptable salt, a hydrate, a solvate, or a polymorph of psilocybin.

19. The kit of claim 15, further comprising a plurality of dosage forms, the plurality comprising one or more doses; wherein each dose comprises a therapeutically effective amount of psilocybin or the pharmaceutical composition thereof.

20. The kit of claim 19, wherein the dosage forms are formulated for oral administration.
